(19) United States
(12) Patent Application Publication
(10) Pub. No.: US 2007/0203384 A1
(43) Pub. Date: Aug. 30, 2007

(54) Oxygenate conversion to olefins with metathesis

(76) Inventors: Peter R. Pujado, Kildeer, IL (US); James M. Andersen, Elmhurst, IL (US)

Correspondence Address: HONEYWELL INTELLECTUAL PROPERTY INC;PATENT SERVICES, 101 COLUMBIA DRIVE, P O BOX 2245 MAIL STOP AB/2B, MORRISTOWN, NJ, 07962, US

(21) Appl. No.: 11/315,935

(22) Filed: Dec. 22, 2005

(51) Int. Cl. C07C 1/00; B01J 8/00

(52) U.S. Cl. 585/639; 422/190

(57) ABSTRACT

Improved processing of an oxygenate-containing feedstock for increased production or yield of light olefins, particularly for increased relative yield of propylene is provided. Such processing involves oxygenate conversion to olefins and subsequent oxygenate conversion effluent stream treatment including isomerization of at least a portion of the 1-butenes to 2-butenes and metathesization of at least a portion of the 2-butenes to produce additional propylene.

### BACKGROUND OF THE INVENTION

This invention relates generally to the conversion of oxygenates to olefins, more particularly, to light olefins.

A major portion of the worldwide petrochemical industry is involved with the production of light olefin materials and their subsequent use in the production of numerous important chemical products via polymerization, oligomerization, alkylation and the like well-known chemical reactions. Light olefins include ethylene, propylene and mixtures thereof. These light olefins are essential building blocks for the modem petrochemical and chemical industries. The major source for these materials in present day refining is the steam cracking of petroleum feeds. For various reasons including geographical, economic, political and diminished supply considerations, the art has long sought a source other than petroleum for the massive quantities of raw materials that are needed to supply the demand for these light olefin materials.

The search for alternative materials for light olefin production has led to the use of oxygenates such as alcohols and, more particularly, to the use of methanol, ethanol, and higher alcohols or their derivatives such as dimethyl ether, diethyl ether, etc., for example. Molecular sieves such as microporous crystalline zeolite and non-zeolitic catalysts, particularly silicoaluminophosphates (SAPO), are known to promote the conversion of oxygenates to hydrocarbon mixtures, particularly hydrocarbon mixtures composed largely of light olefins.

Such processing of oxygenates to form light olefins is commonly referred to as a methanol-to-olefin (MTO) process, as methanol alone or together with other oxygenate materials such as dimethyl ether (DME) is typically an oxygenate material most commonly employed therein. In practice, such oxygenate conversion processing arrangements commonly produce ethylene and propylene as main products and, as stand alone processing, can achieve propylene to ethylene product ratios up to about 1.4. In addition to the production of ethylene and propylene as main products, such processing also typically produces or results in smaller relative amounts of highly olefinic C₄ and heavier hydrocarbon streams.

Commonly assigned, U.S. Pat. No. 5,990,369 to Barger et al., the entire disclosure of which is incorporated herein by reference, discloses a process for the production of light olefins comprising olefins having from 2 to 4 carbon atoms per molecule from an oxygenate feedstock. The process comprises passing the oxygenate feedstock to an oxygenate conversion zone containing a metal aluminophosphate catalyst to produce a light olefin stream. The light olefin stream is fractionated and a portion of the products are metathesized to enhance the yield of the ethylene, propylene, and/or butylene products. Propylene can be metathesized to produce more ethylene, or a combination of ethylene and butene can be metathesized to produce more propylene. The combination of light olefin production and metathesis, or disproportionation is disclosed as providing flexibility such as to overcome the equilibrium limitations of the metal aluminophosphate catalyst in the oxygenate conversion zone. In addition, the invention thereof is disclosed as providing the advantage of extended catalyst life and greater catalyst stability in the oxygenate conversion zone.

While such processing can desirably result in the formation of increased relative amounts of propylene, further improvements such as to further enhance the relative amount of propylene production and recovery are desired and have been sought.

### SUMMARY OF THE INVENTION

A general object of the invention is to provide or result in improved processing of an oxygenate-containing feedstock to light olefins.

A more specific objective of the invention is to overcome one or more of the problems described above.

The general object of the invention can be attained, at least in part, through a specified process for producing light olefins from an oxygenate-containing feedstock. In accordance with one preferred embodiment, such a process involves contacting the oxygenate-containing feedstock in an oxygenate conversion reactor with an oxygenate conversion catalyst and at reaction conditions effective to convert the oxygenate-containing feedstock to form an oxygenate conversion effluent stream comprising light olefins and C₄+ hydrocarbons, wherein the light olefins comprise ethylene and the C₄+ hydrocarbons comprise a quantity of butenes including a quantity of 1-butenes. The oxygenate conversion effluent stream is treated and forms a first process stream comprising at least a portion of the quantity of butenes including 1-butenes from the oxygenate conversion effluent stream. At least a portion of the quantity of 1-butenes of the first process stream are isomerized to form an isomerized stream comprising a quantity of 2-butenes. At least a portion of the quantity of 2-butenes of the isomerized stream are contacted with ethylene in a metathesis zone at effective conditions to produce a metathesis effluent stream comprising propylene with at least a portion of this propylene desirably recovered therefrom.

The prior art generally fails to provide processing schemes and arrangements for the conversion of an oxygenate-containing feedstock to olefins that maximizes production of propylene to as great an extent as may be desired. Moreover, the prior art generally fails to provide a processing scheme and arrangement as effective and efficient as may be desired in increasing the relative yield of propylene in association with the conversion of oxygenate materials to light olefins.

A process for producing light olefins from an oxygenate-containing feedstock in accordance with another embodiment involves contacting an oxygenate-containing feedstock in an oxygenate conversion reactor with an oxygenate conversion catalyst and at reaction conditions effective to convert the oxygenate-containing feedstock to form an oxygenate conversion effluent stream comprising light olefins and C₄+ hydrocarbons. The light olefins desirably include ethylene. The C₄+ hydrocarbons desirably include a quantity of butenes including a quantity of 1-butenes and a quantity of 2-butenes. The oxygenate conversion effluent stream is treated and forms a first process stream consisting essentially of at least a portion of the 1-butenes from the oxygenate conversion effluent stream and a second process stream comprising at least a portion of the ethylene from the oxygenate conversion effluent stream. At least a portion of the 1-butenes of the first process stream are isomerized to form an isomerized stream comprising 2-butenes. In accordance with one particular embodiment, the isomerized stream contains at least 8 moles of 2-butene per mole of 1-butene. At least a portion of the 2-butenes of the isomerized stream are metathesized with at least a portion of the ethylene of the second process stream in a metathesis zone at effective conditions to produce a metathesis effluent stream comprising propylene. Propylene can then be appropriately recovered therefrom.

There is also provided a system for producing light olefins from an oxygenate-containing feedstock. In accordance with one preferred embodiment, such a system includes a reactor for contacting an oxygenate-containing feedstream with an oxygenate conversion catalyst and converting the oxygenate-containing feedstream to form an oxygenate conversion effluent stream comprising light olefins and C₄+ hydrocarbons, wherein the light olefins comprise ethylene and the C₄+ hydrocarbons comprise a quantity of butenes including a quantity of 1-butenes. A treatment zone is provided for treating the oxygenate conversion effluent stream and forming a first process stream comprising at least a portion of the quantity of butenes including 1-butenes from the oxygenate conversion effluent stream. An isomerization zone is provided for isomerizing at least a portion of the quantity of 1-butenes of the first process stream to form an isomerized stream comprising a quantity of 2-butenes. The system for producing light olefins from an oxygenate-containing feedstock further includes a metathesis zone for contacting at least a portion of the quantity of 2-butenes of the isomerized stream with ethylene to produce a metathesis effluent stream comprising propylene. A recovery zone is provided for recovering propylene from the metathesis effluent stream.

As used herein, references to “light olefins” are to be understood to generally refer to C₂ and C₃ olefins, i.e., ethylene and propylene, alone or in combination.

Other objects and advantages will be apparent to those skilled in the art from the following detailed description taken in conjunction with the appended claims and drawings.

### BRIEF DESCRIPTION OF THE DRAWINGS

FIG. 1 is a simplified schematic process flow diagram illustrating a process for the conversion of oxygenates to olefins and employing a butene isomerization zone, to enhance the relative amount of 2-butene, and a metathesis zone, to enhance the yield of propylene, in accordance with one preferred embodiment.

FIG. 2 is a simplified schematic process flow diagram illustrating a process for the conversion of oxygenates to olefins and employing a butene isomerization zone, to enhance the relative amount of 2-butene, and a metathesis zone, to enhance the yield of propylene, in accordance with another preferred embodiment.

FIG. 3 is a simplified schematic process flow diagram illustrating a process for the conversion of oxygenates to olefins and employing a butene isomerization zone, to enhance the relative amount of 2-butene, and a metathesis zone, to enhance the yield of propylene, in accordance with yet another preferred embodiment.

FIG. 4 is a simplified schematic process flow diagram illustrating a process for the conversion of oxygenates to olefins and employing a butene isomerization zone, to enhance the relative amount of 2-butene, and a metathesis zone, to enhance the yield of propylene, in accordance with still yet another preferred embodiment.

### DETAILED DESCRIPTION OF THE INVENTION

Oxygenate-containing feedstock can be converted to light olefins in a catalytic reaction and heavier hydrocarbons (e.g., C₄+ hydrocarbons) formed during such processing can be subsequently treated such that at least a portion of the quantity of 1-butenes formed upon such conversion are subsequently isomerized to form a stream containing 2-butenes. Such 2-butenes can then be metathesized with ethylene to produce additional propylene

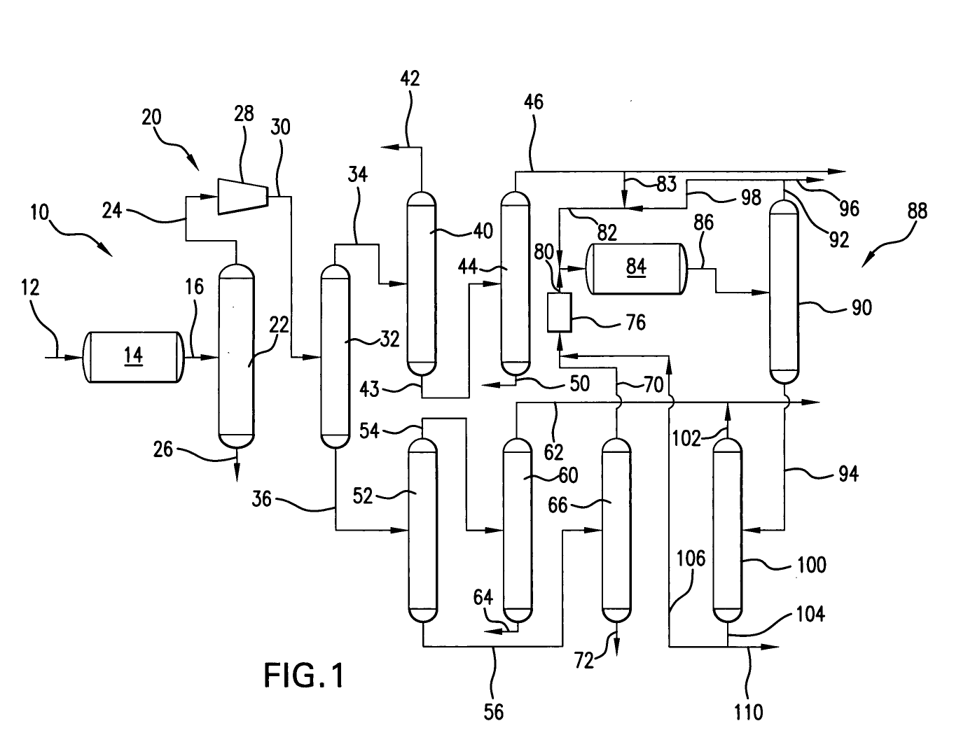

As will be appreciated, such processing may be embodied in a variety of processing arrangements. As representative, FIG. 1 illustrates a simplified schematic process flow diagram for a process scheme, generally designated by the reference numeral 10, for the conversion of oxygenates to olefins and employing a metathesis zone to enhance the yield of propylene, in accordance with one preferred embodiment.

More particularly, an oxygenate-containing feedstock or feedstream 12 such as generally composed of light oxygenates such as one or more of methanol, ethanol, dimethyl ether, diethyl ether, or mixtures thereof, is introduced into an oxygenate conversion zone or reactor section 14 wherein the oxygenate-containing feedstock contacts with an oxygenate conversion catalyst at reaction conditions effective to convert the oxygenate-containing feedstock to form an oxygenate conversion effluent stream comprising fuel gas hydrocarbons, light olefins, and C₄+ hydrocarbons, in a manner as is known in the art, such as, for example, utilizing a fluidized bed reactor.

As will be appreciated by those skilled in the art and guided by the teachings herein provided, such a feedstock may be commercial grade methanol, crude methanol or any methanol purity therebetween. Crude methanol may be an unrefined product from a methanol synthesis unit. Those skilled in that art and guided by the teachings herein provided will understand and appreciate that in the interest of factors such as improved catalyst stability, embodiments utilizing higher purity methanol feeds may be preferred. Thus, suitable feeds in such embodiments may comprise methanol or a methanol and water blend, with possible such feeds having a methanol content of between about 65% and about 100% by weight, preferably a methanol content of between about 80% and about 100% by weight and, in accordance one preferred embodiment, a methanol content of between about 95% and about 100% by weight.

A methanol-to-olefin unit feedstream may comprise between about 0 and about 35 wt-% and more preferably between about 5 and about 30 wt-% water. The methanol in the feed stream may comprise between about 70 and about 100 wt-% and more preferably between about 75 and about 95 wt-% of the feedstream. The ethanol in the feedstream may comprise between about 0.01 and about 0.5 wt-% and more typically between about 0.1 and about 0.2 wt-% of the feedstream although higher concentrations may be beneficial. When methanol is the primary component in the feedstream, the higher alcohols in the feedstream may comprise between about 200 and about 2000 wppm and more typically between about 500 and about 1500 wppm. Additionally, when methanol is the primary component in the feedstream, dimethyl ether in the feedstream may comprise between about 100 and about 20,000 wppm and more typically between about 200 and about 10,000 wppm.

The invention, however, also contemplates and encompasses embodiments wherein the oxygenate-containing feedstock includes dimethyl ether, either alone or in combination with water, methanol or in combination with both water and methanol, for example. The invention specifically encompasses embodiments wherein the oxygenate-containing feedstock is primarily dimethyl ether and, in certain embodiments, wherein the oxygenate-containing feedstock is essentially dimethyl ether, either alone or with no more than insubstantial amounts of other oxygenate materials.

Reaction conditions for the conversion of oxygenates to light olefins are known to those skilled in the art. Preferably, in accordance with particular embodiments, reaction conditions comprise a temperature between about 200° and about 700° C., more preferably between about 300° and 600° C., and most preferably between about 400° and about 550° C. In addition, reactor operating pressures typically are preferably superatmospheric and such as generally range from about 10 psig to about 100 psig (about 69 kPa gauge to about 689 kPa gauge), as may be required to accommodate sufficient pressure at the compressor suction.

As will be appreciated by those skilled in the art and guided by the teachings herein provided, the reactions conditions are generally variable such as dependent on the desired products. For example, if increased ethylene production is desired, then operation at a reactor temperature between about 475° and about 550° C. and more preferably between about 500° and about 520° C., may be preferred. If increased propylene production is desired, then operation at a reactor temperature between about 350° and about 475° C. and more preferably between about 400° and about 430° C. may be preferred. In addition, higher pressures tend to yield slightly more propylene relative to ethylene.

The light olefins produced can have a ratio of ethylene to propylene of between about 0.5 and about 2.0 and preferably between about 0.75 and about 1.25. If a higher ratio of ethylene to propylene is desired, then the reaction temperature is generally desirably higher than if a lower ratio of ethylene to propylene is desired. In accordance with one preferred embodiment, a feed temperature range between about 120° and about 21° C. is preferred. In accordance with another preferred embodiment a feed temperature range of between about 180° and 210° C. is preferred. In accordance with one preferred embodiment, the temperature is desirably maintained below 210° C. to avoid or minimize thermal decomposition.

The oxygenate conversion reactor section 14 produces or results in an oxygenate conversion product or effluent stream 16 such as generally comprising fuel gas hydrocarbons, light olefins, and C₄+ hydrocarbons. The oxygenate conversion effluent stream 16 is passed to an oxygenate conversion effluent stream treatment zone, generally designated by the reference numeral 20. The treatment zone 20 includes a water separation zone 22. In the water separation zone 22, the reactor effluent undergoes separation such as by being quenched with water and then flashed at a separation temperature which is lower than the reactor temperature to provide a vapor effluent stream 24 and a water stream 26. The water stream 26 can be further stripped although not shown in FIG. 1 to remove oxygenates for recycle to the oxygenate conversion reaction zone 14 and the stripped water stream 26 or a portion thereof can be used to generate steam for use in a front-end steam reformer (if a steam reformer is used to generate synthesis gas from natural gas); alternatively, the water may be treated and used for cooling water make-up, irrigation or other desired uses.

The vapor effluent stream 24 may be further processed such as via a compressor section 28 such as composed of one or more compressor stages and, although not shown in the FIG. 1, the vapor effluent stream 24 may be further processed such as by absorbing oxygenates by use of water or methanol absorbent with the absorbent subsequently being stripped of oxygenates to regenerate the absorbent while recycling the oxygenates to the reaction zone 14. The oxygenate-lean olefin product stream may then be conventionally washed with a caustic solution to neutralize any acid gases prior to passage of such a compressed effluent stream 30 to a C₂ fractionation zone 32. In the C₂ fractionation zone 32, the compressed effluent stream 30 is treated, e.g., fractionated, such as by conventional distillation methods, to provide a light ends stream 34 comprising C₂ minus and a C₃ plus stream 36.

The light ends stream 34 is passed to a dernethanizer zone 40. In the demethanizer zone 40, the light ends stream 34 is fractionated such as by conventional distillation methods such as to provide an overhead stream 42 comprising methane and possibly also some inert species (N₂, CO, etc.) and a demethanized C₂ bottoms stream 43 comprising components heavier than methane, such as ethane and ethylene. The stream 42, or a portion thereof and depending on its composition, may be recycled to the front-end unit to make synthesis gas. Alternatively, the stream 42 or a portion thereof can used as fuel.

The demethanized C₂ stream 43 is passed to a C₂ splitter 44. In the C₂ splitter 44, the demethanized C₂ stream 43 is treated, e.g., fractionated, such as by conventional distillation methods, to provide an overhead ethylene product stream 46 such as generally composed of ethylene and a bottoms stream 50 such as generally composed of ethane. Such an ethane-containing bottoms stream or a portion thereof can be recycled to the front-end synthesis gas unit or, if such unit is not readily available or accessible, can be used as fuel.

The C₃ plus stream 36 is passed to a depropanizer zone 52. In the depropanizer zone 52, the C₃ plus stream 36 is treated, e.g., fractionated, such as by conventional distillation methods such as to provide an overhead stream 54 comprising C₃ materials and a depropanized stream 56 generally comprising C₄ plus components. The C₃ materials stream 54 is passed to a C₃ splitter 60. In the C₃ splitter 60, the C₃ materials stream 54 is treated, e.g., fractionated, such as by conventional distillation methods, to provide an overhead propylene product stream 62 such as generally composed of propylene and a bottoms stream 64 such as generally composed of propane. Similar to the above-described ethane-containing bottoms stream, such a propane-containing bottoms stream or a portion thereof can be recycled to the front-end synthesis gas unit or, if such unit is not readily available, can be used as fuel.

The depropanized stream 56 is passed to a C₄ fractionation zone 66. In the C₄ fractionation zone 66, the depropanized stream 56 is fractionated, such as by conventional distillation methods, to provide a mixed butene stream 70, rich in n-butenes and having a low isobutylene content, such as generally composed of 1-butene and 2-butene, such as in an equilibrium mixture, and a C₄ plus stream 72 generally comprising C₄ plus components other than butene.

In general, MTO units produce relatively small amounts of C₅ and heavier compounds. In practice, such a C₄ plus stream or a portion thereof can be used as fuel. For example, for locations in proximity to refineries, such materials or selected portions thereof can be blended into the gasoline pool. Alternatively and such as depending on the specifications as to the olefin content in the feed to the synthesis gas unit, such a C₄ plus stream or a portion thereof can be recycled to the front-end synthesis gas unit.

It has been found that the metathesis reaction of butenes with ethylene over a metathesis catalyst to produce propylene, is favored where the butenes are in the form of 2-butenes rather than 1-butenes. Thus, in accordance with a preferred embodiment, and as described in greater detail below, the mixed butene stream 70, or at least a portion thereof, is passed to an isomerization zone 76 for isomerizing at least a portion of the quantity of 1-butenes therein contained to form an isomerized stream 80 comprising an increased quantity of 2-butenes.

As will be appreciated, such isomerization of 1-butenes to 2-butenes can desirably occur over a suitable isomerization catalyst at selected appropriate isomerization reaction conditions. The 1-butene to 2-butene isomerization reaction is actually a hydroisomerization as it is generally conducted in the presence of a hydrogen atmosphere to facilitate the double bond migration, but such that the use of hydrogen is minimized to avoid undesirable hydrogenation side reactions. The catalysts typically employed in such processing are commonly based on noble metals (palladium, rhodium, platinum, etc.) deposited on an inert alumina support; palladium is normally preferred. Typical or usual reaction conditions may involve a temperature of about 100° to 150° C. and typically a pressure of about 1.5 to 2 MPa (215 to 300 psia). The feed to the hydroisomerization reactor is usually preheated by exchange with the reactor effluent and by steam. Such a heated feed then enters the reactor, which typically operates in a mixed phase with one or more catalyst beds. After cooling, the isomerization products are typically flashed to remove excess hydrogen gas. The reaction temperature is generally chosen so as to maximize conversion to 2-butene (favored by lower temperatures) while still having a reasonable rate of reaction; hence it is commonly desirable to operate at a temperature of less than 150° C. Desirably, the isomerized stream will contain 2-butene and 1-butene in a molar ratio of at least 8, e.g., at least 8 moles of 2-butene per mole of 1-butene, and, in accordance with at least certain preferred embodiments a molar ratio of greater than 10, e.g., more than 10 moles of 2-butene per mole of 1-butene. If fractionated, the residual 1-butene (lighter than 2-butene) can be recycled to the isomerization reactor.

At least a portion of the isomerized stream 80 and a quantity of ethylene, as shown by the process stream 82, such as a portion of the above-described overhead ethylene product stream 46 via line 83, are introduced into a metathesis zone 84 and under effective conditions to produce a metathesis effluent stream 86 comprising propylene.

The metathesis reaction can generally be carried out under conditions and employs catalysts such as are known in the art. In accordance with one preferred embodiment, a metathesis catalyst such as containing a catalytic amount of at least one of molybdenum oxide and tungsten oxide is suitable for the metathesis reaction. Conditions for the metathesis reaction generally include reaction temperature ranging from about 20° to about 450° C., preferably 250° to 350° C., and pressures varying from about atmospheric to upwards of 3,000 psig (20.6 MPa gauge), preferably between 435 and 510 psig (3000 to 3500 kPa gauge), although higher pressures can be employed if desired. Catalysts which are active for the metathesis of olefins and which can be used in the process of this invention are of a generally known type. In this regard, reference is made to “Journal of Molecular Catalysis”, 28 (1984) pages 117-131, to “Journal of Catalysis”, 13 (1969) pages 99-113, to “Applied Catalysis” 10 (1984) pages 29-229 and to “Catalysis Reviews”, 3 (1) 1969) pages 37-60. The disproportionation (metathesis) of 2-butene with ethylene can, for example, be carried out in the vapor phase at about 300° to 350° C. and about 0.5 MPa absolute (75 psia) with a WHSV of 50 to 100 and a once-through conversion of about 15%, depending on the ethylene to 2-butene ratio.

Such metathesis catalysts may be homogeneous or heterogeneous, with heterogeneous catalysts being preferred. The metathesis catalyst preferably comprises a catalytically effective amount of transition metal component. The preferred transition metals for use in the present invention include tungsten, molybdenum, nickel, rhenium, and mixtures thereof. The transition metal component may be present as elemental metal and/or one or more compounds of the metal. If the catalyst is heterogeneous, it is preferred that the transition metal component be associated with a support. Any suitable support material may be employed provided that it does not substantially interfere with the feedstock components or the lower olefin component conversion. Preferably, the support material is an oxide, such as silica, alumina, titania, zirconia and mixtures thereof. Silica is a particularly preferred support material. If a support material is employed, the amount of transition metal component used in combination with the support material may vary widely depending, for example, on the particular application involved and/or the transition metal being used. Preferably, the transition metal comprises about 1% to about 20%, by weight (calculated as elemental metal) of the total catalyst. The metathesis catalyst may advantageously comprise a catalytically effective amount of at least one of the above-noted transition metals, and are capable of promoting olefin metathesis. The catalyst may also contain at least one activating agent present in an amount to improve the effectiveness of the catalyst. Various activating agents may be employed, including activating agents which are well known in the art to facilitate metathesis reactions. Light olefin metathesis catalysts can, for example, desirably be complexes of tungsten (W), molybdenum (Mo), or rhenium (Re) in a heterogeneous or homogeneous phase.

In general, the metathesis equilibrium for propylene production is also favored by lower temperatures and higher ethylene:2-butene ratios. For example, at a temperature of 600 K., the metathesis equilibria shown in the following Table, below, can be established:

| Ethylene:2-Butene Ratio | 2-Butene Converted (mol-%) |
|---|---|
| 1 | 65 |
| 2 | 83 |
| 3 | 89 |

The metathesis effluent stream 86 is passed to a metathesis effluent treatment zone 88 such as includes an ethylene column 90 wherein ethylene may be separated from the balance of the metathesis effluent to form an ethylene stream 92 and a balance of the metathesis effluent stream 94. The ethylene stream 92 can, in whole or in part, be passed or forwarded, as shown by the line 98 and the line 82, to the metathesis zone 84 such as for metathesis with butene. A purge stream 96 may be provided to avoid buildup of impurities or inert species in the ethylene recycle loop.

The balance of the metathesis effluent stream 94 is passed to a propylene column 100. In the propylene column 100, the balance of the metathesis effluent stream is treated, e.g., fractionated, such as by conventional distillation methods, to provide an overhead propylene product stream 102 such as generally composed of propylene and a bottoms stream 104 such as to form a butene stream 106 such as can be returned for further metathesis processing and a C₄ purge stream 110 such as can desirably be included to avoid undesired buildup of heavies or other nonreacting materials (e.g., saturates) that might otherwise accumulate in the loop. The purge stream 110 can desirably go to fuel.

In the embodiment shown in FIG. 1, both the butene stream 70 resulting from the C₄ fractionation zone 66 and the butene stream 106 resulting from the propylene column 100 of the metathesis effluent treatment zone 88 are passed into the isomerization zone 76 for isomerizing at least a portion of the quantity of 1-butenes therein contained to form the isomerized stream 80 comprising an increased quantity of 2-butenes. Those skilled in the art and guided by the teachings herein provided will, however, appreciate that the broader practice of the invention is not necessarily so limited.

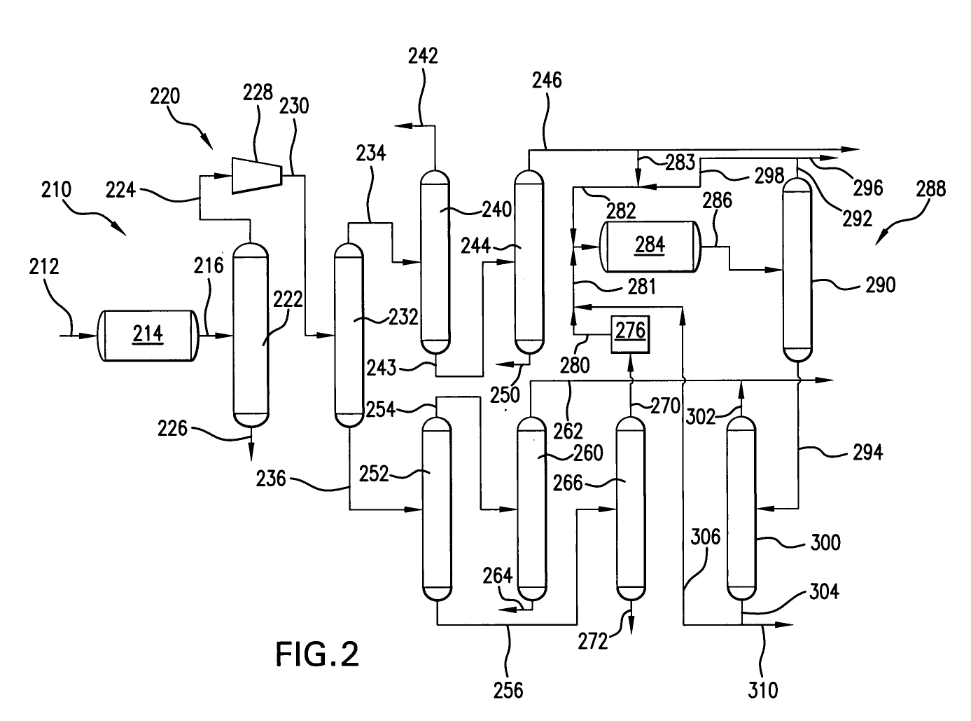

For example, in alternative embodiment, it may be desirable that only the butene stream resulting from such a C₄ fractionation zone, such butene hereinafter sometimes referred to as “fresh butene”, be subjected to such isomerization prior to metathesis. A simplified schematic process flow diagram for such a process scheme, generally designated by the reference numeral 210 is generally shown in FIG. 2

The process scheme 210 is generally similar to the process scheme 10 described above and having an oxygenate-containing feedstock or feedstream 212, such as described above, that is introduced into an oxygenate conversion zone or reactor section 214 wherein the oxygenate-containing feedstock contacts with an oxygenate conversion catalyst at reaction conditions effective to convert the oxygenate-containing feedstock and to form an oxygenate conversion effluent stream comprising fuel gas hydrocarbons, light olefins, and C₄+ hydrocarbons, in a manner as is known in the art, such as, for example, utilizing a fluidized bed reactor.

The oxygenate conversion reactor section 214 produces or results in an oxygenate conversion product or effluent stream 216 such as generally comprising fuel gas hydrocarbons, light olefins, and C₄+ hydrocarbons. The oxygenate conversion effluent stream 216 is passed to an oxygenate conversion effluent stream treatment zone, generally designated by the reference numeral 220. The treatment zone 220 includes a water separation zone 222 wherein the reactor effluent undergoes separation such as by being quenched with water and then flashed at a separation temperature which is lower than the reactor temperature to provide a vapor effluent stream 224 and a water stream 226. The water stream 226 can be further stripped although not shown in FIG. 2 to remove oxygenates for recycle to the oxygenate conversion reaction zone 214.

The vapor effluent stream 224 may be further processed such as via a compressor section 228 such as composed of one or more compressor stages. As with the process scheme 10 described above, the vapor effluent stream 224 may be further processed such as by being conventionally washed with a caustic solution to neutralize any acid gases and remove catalyst fines and dried prior to passage of such a compressed effluent stream 230 to a C₂ fractionation zone 232. In the C₂ fractionation zone 232, the compressed effluent stream 230 is treated, e.g., fractionated, such as by conventional distillation methods, to provide a light ends stream 234 comprising C₂ minus and a C₃ plus stream 236.

The light ends stream 234 is passed to a demethanizer zone 240. In the demethanizer zone 240, the light ends stream 234 is fractionated such as by conventional distillation methods such as to provide an overhead stream 242 comprising methane and a demethanized C₂ bottoms stream 243 comprising components heavier than methane, such as ethane and ethylene. The demethanized C₂ stream 243 is passed to a C₂ splitter 244. In the C₂ splitter 244, the demethanized C₂ stream 243 is treated, e.g., fractionated, such as by conventional distillation methods, to provide an overhead ethylene product stream 246 such as generally composed of ethylene and a bottoms stream 250 such as generally composed of ethane.

The C₃ plus stream 236 is passed to a depropanizer zone 252. In the depropanizer zone 252, the C₃ plus stream 236 is treated, e.g., fractionated, such as by conventional distillation methods such as to provide an overhead stream 254 comprising C₃ materials and a depropanized stream 256 generally comprising C₄ plus components. The C₃ materials stream 254 is passed to a C₃ splitter 260. In the C₃ splitter 260, the C₃ materials stream 254 is treated, e.g., fractionated, such as by conventional distillation methods, to provide an overhead propylene product stream 262 such as generally composed of propylene and a bottoms stream 264 such as generally composed of propane.

The depropanized stream 256 is passed to a C₄ fractionation zone 266. In the C₄ fractionation zone 266, the depropanized stream 256 is fractionated, such as by conventional distillation, methods to provide a mixed butene stream 270, such as generally composed of 1-butene and 2-butene, such as in an equilibrium mixture, and a C₄ plus stream 272 generally comprising C₄ plus components other than butene.

The mixed butene stream 270, or at least a portion thereof, is passed to an isomerization zone 276 for isomerizing, such as described above, at least a portion of the quantity of 1-butenes therein contained to form an isomerized stream 280 comprising an increased quantity of 2-butenes.

At least a portion of the isomerized stream 280, such as via a line 281, and a quantity of ethylene, as shown by the process stream 282, such as a portion of the above-described overhead ethylene product stream 246 via line 283, are introduced into a metathesis zone 284 and under effective conditions to produce a metathesis effluent stream 286 comprising propylene.

The metathesis effluent stream 286 is passed to a metathesis effluent treatment zone 288 such as includes an ethylene column 290 wherein ethylene may be separated from the balance of the metathesis effluent to form an ethylene stream 292 and a balance of the metathesis effluent stream 294. The ethylene stream 292 can, in whole or in part, be passed or forwarded, as shown by the line 298 and the line 282, to the metathesis zone 284 such as for metathesis with butene. A purge stream 296 may be provided to avoid buildup of impurities or inert species in the ethylene recycle loop.

The balance of the metathesis effluent stream 294 is passed to a propylene column 300. In the propylene column 300, the balance of the metathesis effluent stream is treated, e.g., fractionated, such as by conventional distillation methods, to provide an overhead propylene product stream 302 such as generally composed of propylene and a bottoms stream 304 such as to form a butene stream 306 such as can be returned for further metathesis processing and a C₄ purge stream 310 such as can desirably be included to avoid undesired buildup of heavies or other nonreacting materials (e.g., saturates) that might otherwise accumulate in the loop. The purge stream 310 can desirably go to fuel.

As shown, in this embodiment, the butene stream 306 is returned, such as via the line 281, for further metathesis processing without first being subjected to isomerization processing. Alternatively, the propylene resulting from such metathesis processing may already meet polymer-grade specifications such that such a stream no longer requires the inclusion of such a C₃ splitter column.

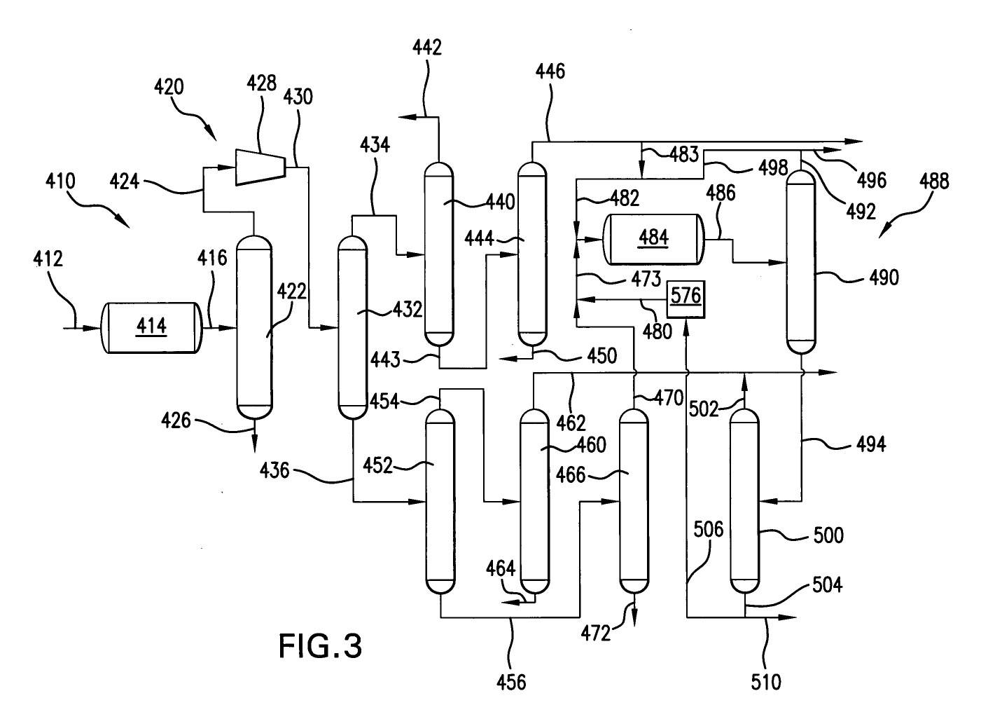

While the processing schemes 10 and 210, shown in FIGS. 1 and 2 and described above, involved isomerization treatment of fresh butene, in accordance with another preferred embodiment, it may be desirable that only butenes resulting from such a metathesis treatment zone, such butene hereinafter sometimes referred to as “recycle butene”, be subjected to such isomerization prior to metathesis. A simplified schematic process flow diagram for such a process scheme, generally designated by the reference numeral 410 is generally shown in FIG. 3.

The process scheme 410 is generally similar to the process scheme 10 described above and having an oxygenate-containing feedstock or feedstream 412, such as described above, that is introduced into an oxygenate conversion zone or reactor section 414 wherein the oxygenate-containing feedstock contacts with an oxygenate conversion catalyst at reaction conditions effective to convert the oxygenate-containing feedstock to form an oxygenate conversion effluent stream comprising fuel gas hydrocarbons, light olefins, and C₄+ hydrocarbons, in a manner as is known in the art, such as, for example, utilizing a fluidized bed reactor.

The oxygenate conversion reactor section 414 produces or results in an oxygenate conversion product or effluent stream 416 such as generally comprising fuel gas hydrocarbons, light olefins, and C₄+ hydrocarbons. The oxygenate conversion effluent stream 416 is passed to an oxygenate conversion effluent stream treatment zone, generally designated by the reference numeral 420. The treatment zone 420 includes a water separation zone 422 wherein the reactor effluent undergoes separation such as by being quenched with water and then flashed at a separation temperature which is lower than the reactor temperature to provide a vapor effluent stream 424 and a water stream 426. The water stream 426 can be further stripped although not shown in FIG. 3 to remove oxygenates for recycle to the oxygenate conversion reaction zone 414.

The vapor effluent stream 424 may be further processed such as via a compressor section 428 such as composed of one or more compressor stages. As with the process scheme 10 described above, the vapor effluent stream 424 may be further processed such as by being conventionally washed with a caustic solution to neutralize any acid gases and remove catalyst fines and dried prior to passage of such a compressed effluent stream 430 to a C₂ fractionation zone 432. In the C₂ fractionation zone 432, the compressed effluent stream 430 is treated, e.g., fractionated, such as by conventional distillation methods, to provide a light ends stream 434 comprising C₂ minus and a C₃ plus stream 436.

The light ends stream 434 is passed to a demethanizer zone 440. In the demethanizer zone 440, the light ends stream 434 is fractionated such as by conventional distillation methods such as to provide an overhead stream 442 comprising methane and a demethanized C₂ bottoms stream 443 comprising components heavier than methane, such as ethane and ethylene. The demethanized C₂ stream 443 is passed to a C₂ splitter 444. In the C₂ splitter 444, the demethanized C₂ stream 443 is treated, e.g., fractionated, such as by conventional distillation methods, to provide an overhead ethylene product stream 446 such as generally composed of ethylene and a bottoms stream 450 such as generally composed of ethane.

The C₃ plus stream 436 is passed to a depropanizer zone 452. In the depropanizer zone 452, the C₃ plus stream 436 is treated, e.g., fractionated, such as by conventional distillation methods such as to provide an overhead stream 454 comprising C₃ materials and a depropanized stream 456 generally comprising C₄ plus components. The C₃ materials stream 454 is passed to a C₃ splitter 460. In the C₃ splitter 460, the C₃ materials stream 454 is treated, e.g., fractionated, such as by conventional distillation methods, to provide an overhead propylene product stream 462 such as generally composed of propylene and a bottoms stream 464 such as generally composed of propane.

The depropanized stream 456 is passed to a C₄ fractionation zone 466. In the C₄ fractionation zone 466, the depropanized stream 456 is fractionated, such as by conventional distillation, methods to provide a mixed butene stream 470, such as generally composed of 1-butene and 2-butene, such as in an equilibrium mixture, and a C₄ plus stream 472 generally comprising C₄ plus components other than butene.

In this embodiment, the mixed butene stream 470, or at least a portion thereof such as via a line 473, and a quantity of ethylene, as shown by the process stream 482, such as a portion of the above-described overhead ethylene product stream 446 via a line 483, are introduced into a metathesis zone 484 and under effective conditions to produce a metathesis effluent stream 486 comprising propylene.

The metathesis effluent stream 486 is passed to a metathesis effluent treatment zone 488 such as includes an ethylene column 490 wherein ethylene may be separated from the balance of the metathesis effluent to form an ethylene stream 492 and a balance of the metathesis effluent stream 494. The ethylene stream 492 can, in whole or in part, be passed or forwarded, as shown by the line 498 and the line 482, to the metathesis zone 484 such as for metathesis with butene. A purge stream 496 may be provided to avoid buildup of impurities or inert species in the ethylene recycle loop. The balance of the metathesis effluent stream 494 is passed to a propylene column 500. In the propylene column 500, the balance of the metathesis effluent stream is treated, e.g., fractionated, such as by conventional distillation methods, to provide an overhead propylene product stream 502 such as generally composed of propylene and a bottoms stream 504 such as to form a butene stream 506 and a C₄ purge stream 510.

As shown in FIG. 3, in this embodiment, the butene stream 506 is passed to an isomerization zone 576 for isomerizing, such as described above, such that at least a portion of the quantity of 1-butenes therein contained are isomerized to form an isomerized stream 480 comprising an increased quantity of 2-butenes.

The mixed butene stream 470 and the isomerized stream 480, such as via the line 473 and quantity of ethylene, as shown by the process stream 482, such as a portion of the above-described overhead ethylene product stream 446, are introduced into a metathesis zone 484 and under effective conditions to produce a metathesis effluent stream 486 comprising propylene.

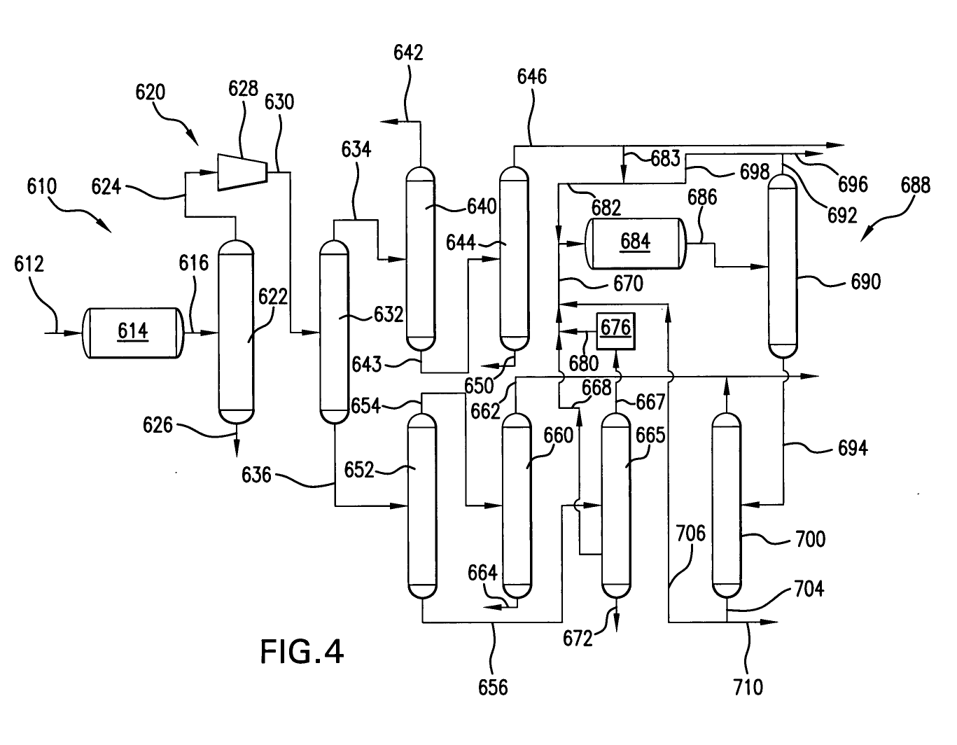

FIG. 4 illustrates a process scheme, generally designated by the reference numeral 610, for the conversion of oxygenates to olefins and employing a butene isomerization zone, to enhance the relative amount of 2-butene, and a metathesis zone, to enhance the yield of propylene, in accordance with still yet another preferred embodiment.

The process scheme 610, similar to the process scheme 10 described above, utilizes an oxygenate-containing feedstock or feedstream 612, such as described above, that is introduced into an oxygenate conversion zone or reactor section 614 wherein the oxygenate-containing feedstock contacts with an oxygenate conversion catalyst at reaction conditions effective to convert the oxygenate-containing feedstock to form an oxygenate conversion effluent stream comprising fuel gas hydrocarbons, light olefins, and C₄+ hydrocarbons, in a manner as is known in the art, such as, for example, utilizing a fluidized bed reactor.

The oxygenate conversion reactor section 614 produces or results in an oxygenate conversion product or effluent stream 616 such as generally comprising fuel gas hydrocarbons, light olefins, and C₄+ hydrocarbons. The oxygenate conversion effluent stream 616 is passed to an oxygenate conversion effluent stream treatment zone, generally designated by the reference numeral 620. The treatment zone 620 includes a water separation zone 622 wherein the reactor effluent undergoes separation such as by being quenched with water and then flashed at a separation temperature which is lower than the reactor temperature to provide a vapor effluent stream 624 and a water stream 626. The water stream 626 can be further stripped although not shown in FIG. 4 to remove oxygenates for recycle to the oxygenate conversion reaction zone 614.

The vapor effluent stream 624 may be further processed such as via a compressor section 628 such as composed of one or more compressor stages. As with the process scheme 10 described above, the vapor effluent stream 624 may be further processed such as by being conventionally washed with a caustic solution to neutralize any acid gases and remove catalyst fines and dried prior to passage of such a compressed effluent stream 630 to a C₂ fractionation zone 632. In the C₂ fractionation zone 632, the compressed effluent stream 630 is treated, e.g., fractionated, such as by conventional distillation methods, to provide a light ends stream 634 comprising C₂ minus and a C₃ plus stream 636.

The light ends stream 634 is passed to a demethanizer zone 640. In the demethanizer zone 640, the light ends stream 634 is fractionated such as by conventional distillation methods such as to provide an overhead stream 642 comprising methane and a demethanized C₂ bottoms stream 643 comprising components heavier than methane, such as ethane and ethylene. The demethanized C₂ stream 643 is passed to a C₂ splitter 644. In the C₂ splitter 644, the demethanized C₂ stream 643 is treated, e.g., fractionated, such as by conventional distillation methods, to provide an overhead ethylene product stream 646 such as generally composed of ethylene and a bottoms stream 650 such as generally composed of ethane.

The C₃ plus stream 636 is passed to a depropanizer zone 652. In the depropanizer zone 652, the C₃ plus stream 636 is treated, e.g., fractionated, such as by conventional distillation methods such as to provide an overhead stream 654 comprising C₃ materials and a depropanized stream 656 generally comprising C₄ plus components. The C₃ materials stream 654 is passed to a C₃ splitter 660. In the C₃ splitter 660, the C₃ materials stream 654 is treated, e.g., fractionated, such as by conventional distillation methods, to provide an overhead propylene product stream 662 such as generally composed of propylene and a bottoms stream 664 such as generally composed of propane.

The depropanized stream 656 is passed to a C₄ superfractionation zone 665. In the C₄ superfractionation zone 665, the depropanized stream 656 is superfractionated to form a stream 667 composed primarily of 1-butene, a residual stream 668 of butenes, having a high relative content of 2-butenes and a C₄ plus stream 672 generally comprising C₄ plus components other than butene. In accordance with the illustrated embodiment, such a residual stream 668 can desirably be sent, such as via the lines 669 and 670, to the metathesis zone 684. The 1-butene stream 667 can be sent to an isomerization zone 676, such as described above, for isomerization of at least a portion of the quantity of 1-butenes therein contained to form an isomerized stream 680 comprising an increased quantity of 2-butenes, with at least a portion of the isomerized stream 680 being introduced into the metathesis zone 684, such as via the lines 669 and 670.

A quantity of ethylene, as shown by the process stream 682, such as a portion of the above-described overhead ethylene product stream 646 via a line 683, is also introduced into the metathesis zone 684 and under effective conditions to produce a metathesis effluent stream 686 comprising propylene.

The metathesis effluent stream 686 is passed to a metathesis effluent treatment zone 688 such as includes an ethylene column 690 wherein ethylene may be separated from the balance of the metathesis effluent to form an ethylene stream 692 and a balance of the metathesis effluent stream 694. The ethylene stream 692 can, in whole or in part, be passed or forwarded, as shown by the line 698 and the line 682, to the metathesis zone 684 such as for metathesis with butene. A purge stream 696 may be provided to avoid buildup of impurities or inert species in the ethylene recycle loop.

The balance of the metathesis effluent stream 694 is passed to a propylene column 700. In the propylene column 700, the balance of the metathesis effluent stream is treated, e.g., fractionated, such as by conventional distillation methods, to provide an overhead propylene product stream 702 such as generally composed of propylene and a bottoms stream 704 such as to form a butene stream 706 such as can be returned for further metathesis processing and a C₄ purge stream 710. As shown, in this embodiment, the butene stream 706 can be returned to metathesis zone 684, such as via the line 670, for further metathesis processing without first being subjected to isomerization processing. However, it is also contemplated to direct the butane stream 706 to the isomerization reactor 676 along with the 1-butene stream 667 similar to the embodiment of FIG. 1.

Thus, through the application of butene isomerization and metathesis of butenes with ethylene, such as described above, there are provided processes and systems for the conversion of an oxygenate-containing feedstock to olefins that maximizes production of propylene to a greater extent than heretofore practically realizable. Moreover, processing schemes and arrangements are provided that are desirably effective and efficient in increasing the relative yield of propylene in association with oxygenate conversion of light olefins. In particular, the processing and system integration of the conversion of oxygenates to olefins with metathesis, as described herein, can desirably result in achieving propylene to ethylene product ratios of at least two or more and, in accordance with at least certain embodiments, processing as herein described can desirably result in achieving propylene to ethylene product ratios of at least 2.3 or more. In particular embodiments, the processing and system integration of the conversion of oxygenates to olefins with metathesis can desirably be combined with high pressure, low temperature operation such that propylene to ethylene product ratios of at least 3 to 4, such as propylene to ethylene product ratios in the range of 4 to 5 can be obtained and realized.

The invention illustratively disclosed herein suitably may be practiced in the absence of any element, part, step, component, or ingredient which is not specifically disclosed herein.

While in the foregoing detailed description this invention has been described in relation to certain preferred embodiments thereof, and many details have been set forth for purposes of illustration, it will be apparent to those skilled in the art that the invention is susceptible to additional embodiments and that certain of the details described herein can be varied considerably without departing from the basic principles of the invention.

What is claimed is:

1. A process for producing light olefins from an oxygenate-containing feedstock, said process comprising:
contacting the oxygenate-containing feedstock in an oxygenate conversion reactor with an oxygenate conversion catalyst and at reaction conditions effective to convert the oxygenate-containing feedstock to an oxygenate conversion effluent stream comprising light olefins and C₄+ hydrocarbons, wherein the light olefins comprise ethylene and the C₄+ hydrocarbons comprise a quantity of butenes including a quantity of 1-butenes;
treating the oxygenate conversion effluent stream and forming a first process stream comprising at least a portion of the quantity of butenes including 1-butenes from the oxygenate conversion effluent stream;
isomerizing at least a portion of the quantity of 1-butenes of the first process stream to form an isomerized stream comprising a quantity of 2-butenes;
contacting at least a portion of the quantity of 2-butenes of the isomerized stream with ethylene in a metathesis zone at effective conditions to produce a metathesis effluent stream comprising propylene; and
recovering propylene from the metathesis effluent stream.

2. The process of claim 1 wherein the treating step additionally forms a second process stream comprising at least a portion of the ethylene from the oxygenate conversion effluent stream and wherein at least a portion of the ethylene of the second process stream is introduced into the metathesis zone to metathesize with at least a portion of the quantity of 2-butenes to produce propylene.

3. The process of claim 1 wherein the C₄+ hydrocarbons of the oxygenate conversion effluent stream additionally comprises a quantity of 2-butenes and wherein during said metathesis step, at least a portion of said quantity of 2-butenes is also metathesized with ethylene in the metathesis zone at effective conditions to produce additional propylene included in the metathesis effluent stream.

4. The process of claim 3 wherein the treating step additionally forms a second process stream comprising at least a portion of the ethylene from the oxygenate conversion effluent stream and wherein at least a portion of the ethylene of the second process stream is introduced into the metathesis zone to metathesize with at least a portion of the quantity of 2-butenes to produce propylene.

5. The process of claim 1 wherein the C₄+ hydrocarbons of the oxygenate conversion effluent stream additionally comprise a quantity of 2-butenes and wherein said process additionally comprises separating 1-butenes from 2-butenes prior to isomerization of the separated 1-butenes.

6. The process of claim 5 wherein the treating step additionally forms a second process stream comprising at least a portion of the ethylene from the oxygenate conversion effluent stream and wherein at least a portion of the ethylene of the second process stream is introduced into the metathesis zone to metathesize with at least a portion of the quantity of 2-butenes to produce propylene.

7. The process of claim 1 wherein the metathesis effluent stream additionally comprises a quantity of butenes, said process additionally comprising:
separating at least a portion of the quantity of butenes from the metathesis effluent stream, and
recycling at least a portion of the separated butenes to the metathesis zone.

8. The process of claim 7 wherein said isomerization of at least a portion of the 1-butenes of the first process stream comprises isomerization of the recycled portion of the separated butenes.

9. The process of claim 5 wherein the treating step additionally forms a second process stream comprising at least a portion of the ethylene from the oxygenate conversion effluent stream and wherein at least a portion of the ethylene of the second process stream is introduced into the metathesis zone to metathesize with at least a portion of the quantity of 2-butenes to produce propylene.

10. The process of claim 1 wherein said isomerizing results in an isomerized stream comprising at least 8 moles of 2-butene per mole of 1-butene.

11. A process for producing light olefins from an oxygenate-containing feedstock, said process comprising:
contacting the oxygenate-containing feedstock in an oxygenate conversion reactor with an oxygenate conversion catalyst and at reaction conditions effective to convert the oxygenate-containing feedstock to an oxygenate conversion effluent stream comprising light olefins and C₄+ hydrocarbons, wherein the light olefins comprise ethylene and the C₄+ hydrocarbons comprise a quantity of butenes including a quantity of 1-butenes and a quantity of 2-butenes;
treating the oxygenate conversion effluent stream and forming a first process stream consisting essentially of at least a portion of the 1-butenes from the oxygenate conversion effluent stream and a second process stream comprising at least a portion of the ethylene from the oxygenate conversion effluent stream;
isomerizing at least a portion of the 1-butenes of the first process stream to form an isomerized stream comprising 2-butenes, wherein the isomerized stream contains at least 8 moles of 2-butene per mole of 1-butene;
metathesizing at least a portion of the 2-butenes of the isomerized stream with at least a portion of the ethylene of the second process stream in a metathesis zone at effective conditions to produce a metathesis effluent stream comprising propylene; and
recovering propylene from the metathesis effluent stream.

12. The process of claim 11 wherein the C₄+ hydrocarbons of the oxygenate conversion effluent stream additionally consists essentially of a quantity of 2-butenes and wherein during said metathesis step, at least a portion of said quantity of 2-butenes is also metathesized with ethylene in the metathesis zone at effective conditions to produce additional propylene included in the metathesis effluent stream.

13. The process of claim 11 wherein the C₄+ hydrocarbons of the oxygenate conversion effluent stream additionally comprise a quantity of 2-butenes and wherein said process additionally comprises separating 1-butenes from 2-butenes prior to isomerization of the separated 1-butenes.

14. The process of claim 11 wherein the metathesis effluent stream additionally comprises a quantity of butenes, said process additionally comprising:
separating at least a portion of the quantity of butenes from the metathesis effluent stream, and
recycling at least a portion of the separated butenes to the metathesis zone.

15. The process of claim 14 wherein said isomerization of at least a portion of the 1-butenes of the first process stream comprises isomerization of the recycled portion of the separated butenes.

16. A system for producing light olefins from an oxygenate-containing feedstock, said system comprising:
a reactor for contacting an oxygenate-containing feedstream with an oxygenate conversion catalyst and converting the oxygenate-containing feedstream to an oxygenate conversion effluent stream comprising light olefins and C₄+ hydrocarbons, wherein the light olefins comprise ethylene and the C₄+ hydrocarbons comprise a quantity of butenes including a quantity of 1-butenes;
a treatment zone for treating the oxygenate conversion effluent stream and forming a first process stream comprising at least a portion of the quantity of butenes including 1-butenes from the oxygenate conversion effluent stream;
an isomerization zone for isomerizing at least a portion of the quantity of 1-butenes of the first process stream to form an isomerized stream comprising a quantity of 2-butenes;
a metathesis zone for contacting at least a portion of the quantity of 2-butenes of the isomerized stream with ethylene to produce a metathesis effluent stream comprising propylene; and
a recovery zone for recovering propylene from the metathesis effluent stream.

17. The system of claim 16 wherein the treatment zone comprises a plurality of fractionation zones.

18. The system of claim 17 wherein the plurality of fractionation zones comprises a C₄ fractionation zone effective to provide a mixed butene stream.

19. The system of claim 18 wherein the isomerization zone is interposed between the C₄ fractionation zone and the metathesis zone.

20. The system of claim 16 wherein the recovery zone is also effective in recovering butenes and transmitting said recovered butenes to the metathesis zone.

21. The system of claim 20 wherein the isomerization zone is interposed between the recovery zone and the metathesis zone.
